SEQUENCE LISTING (17 sequences)

SEQ ID NO: 1 - protein, 124 aa, Artificial sequence
  Synthetic Amino Acids
ASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYA

SEQ ID NO: 2 - protein, 112 aa, Artificial sequence
  Synthetic Amino Acids
AEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTF

SEQ ID NO: 3 - protein, 8 aa, Artificial sequence
  Synhetic Amino Acids
PRQKRTAT

SEQ ID NO: 4 - protein, 21 aa, Artificial sequence
  Synhetic Amino Acids
SSRSSSRSRNSSRNSTPGSSR

SEQ ID NO: 5 - protein, 4 aa, Artificial sequence
  Synhetic Amino Acids
GGGS

SEQ ID NO: 6 - protein, 8 aa, Artificial sequence
  Synhetic Amino Acids
GGSGGGGS

SEQ ID NO: 7 - protein, 10 aa, Artificial sequence
  Synhetic Amino Acids
HHHHHHHHHH

SEQ ID NO: 8 - protein, 6 aa, Artificial sequence
  Synhetic Amino Acids
ENLYFQ

SEQ ID NO: 9 - protein, 419 aa, Artificial sequence
  Synhetic Amino Acids
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 10 - protein, 420 aa, Artificial sequence
  Synhetic Amino Acids
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQAGGSGGGGSGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGSHHHHHHHHHH

SEQ ID NO: 11 - protein, 428 aa, Artificial sequence
  Synhetic Amino Acids
MSHHHHHHHHHHGGGSENLYFQMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQAGGSGGGGSGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 12 - protein, 196 aa, Artificial sequence
  Synhetic Amino Acids
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQAGGGSHHHHHHHHHH

SEQ ID NO: 13 - protein, 204 aa, Artificial sequence
  Synhetic Amino Acids
MSHHHHHHHHHHGGGSENLYFQMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQA

SEQ ID NO: 14 - protein, 184 aa, Artificial sequence
  Synhetic Amino Acids
MSAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGSHHHHHHHHHH

SEQ ID NO: 15 - protein, 190 aa, Artificial sequence
  Synhetic Amino Acids
MSHHHHHHHHHHGGGSENLYFQAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 16 - protein, 249 aa, Artificial sequence
  Synhetic Amino Acids
MSASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGSGGGGSAEASKKNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFGGGSHHHHHHHHHH

SEQ ID NO: 17 - protein, 255 aa, Artificial sequence
  Synhetic Amino Acids
MSHHHHHHHHHHGGGSENLYFQASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAGGSGGGGSAEASKKNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTF

(19) United States
(12) Patent Application Publication
(10) Pub. No.: US 2023/0303629 A1
(43) Pub. Date: Sep. 28, 2023

(54) FUSION PROTEINS COMPRISING SARS-COV-2 NUCLEOCAPSID DOMAINS

(71) Applicant: GRIFOLS DIAGNOSTIC SOLUTIONS INC., Emeryville, CA (US)

(72) Inventors: EFRAIN CEH PAVIA, Emeryville, CA (US); ELISABETE NASCIMENTO, Emeryville, CA (US); ELIZABETH A. BOOTH, Emeryville, CA (US); ZEBULON LAPOINT, Emeryville, CA (US); CHARLES HOLZ, Emeryville, CA (US); TRISTAN WASLEY, Emeryville, CA (US); JODY MELTON WITT, Emeryville, CA (US)

(21) Appl. No.: 18/020,797

(22) PCT Filed: Aug. 17, 2021
(86) PCT No.: PCT/IB2021/057543

§ 371 Date: Feb. 10, 2023

Related U.S. Application Data
(60) Provisional application No. 63/066,680, filed on Aug. 17, 2020.

(51) Int. Cl. C07K 14/005; C07K 17/00; C07K 1/107; C12N 7/00

(52) U.S. Cl. CPC C07K 14/005; C07K 17/00; C07K 1/1075; C12N 7/00; C07K 2319/00; C12N 2770/20022; C12N 2770/20051

(57) ABSTRACT

A fusion protein includes a SARS-CoV-2 nucleocapsid N-terminal domain and/or a SARS-CoV-2 nucleocapsid C-terminal domain, wherein the fusion protein lacks the SARS-CoV-2 nucleocapsid aggregation domain.

### PRIORITY AND CROSS REFERENCE TO RELATED APPLICATIONS

This application is the U.S. National Phase application under 35 U.S.C. § 371 of International Application No. PCT/IB2021/057543, filed Aug. 17, 2021, designating the U.S. and published as WO 2022/038499 A1 on Feb. 24, 2022, which claims the benefit of Provisional Application No. 63/066,680, filed Aug. 17, 2020. Any and all applications for which a foreign or a domestic priority is claimed is/are identified in the Application Data Sheet filed herewith and is/are hereby incorporated by reference in their entireties under 37 C.F.R. § 1.57.

### SEQUENCE LISTING

This application is being filed along with a Sequence Listing in electronic format. The Sequence Listing is provided as a file entitled SeqList-DURC048.011APC, created Jan. 23, 2023, which is approximately 25 kilobytes in size. The information in the electronic format of the Sequence Listing is incorporated herein by reference in its entirety.

### FIELD

This application relates to the medical field of COVID-19 diagnosis or treatment, and in particular, it relates to fusion proteins comprising severe acute respiratory syndrome coronavirus 2 (SARS-CoV-2) nucleocapsid domains or fragments thereof. Said fusion proteins are useful for the development of assays for detection of SARS-CoV-2.

### BACKGROUND

SARS-CoV-2 is an enveloped RNA virus from the Coronaviridae family (Gorbalenya, A. E., et al., 2020, Nature Microbiology, 5(4):p. 536-544). There are four structural proteins in SARS-CoV-2: spike (S), nucleocapsid (N), envelope (E) and membrane (M) proteins (Lu, R., et al., 2020, Lancet, 395(10224):p. 565-574). Of these, S and N have been shown to be the most immunogenic.

SARS-CoV-2 has caused a widespread COVID-19 pandemic that infected millions worldwide and claimed hundreds of thousands of lives. Currently, the main and most accurate method of diagnosis is by RT-PCR testing of nasopharyngeal swabs (Peng et al., 2020, J Med Virol. 24; 10.1002/jmv.25936). However, a viral assay can only identify active SARS-CoV-2 infections but provides no evidence of past infections, particularly in asymptomatic patients.

To obtain a more accurate account of the SARS-CoV-2 infection level in a population it is necessary to employ serological screening. A serological test looks for the presence of antibodies in patient samples (serum or plasma). These antibodies arise in response to a specific infection and can be found in patients days after viral clearance. Yet, there is an urgent need to develop reliable, highly sensitive and specific antibody tests capable of identifying all infected individuals, irrespective of clinical symptoms. This information will be critical to establish community surveillance and implement policies that contain the viral spread.

The US Food and Drug Administration (FDA) has granted Emergency Use Authorizations (EUA) to multiple immunoassay tests in the market, but none of those assays has been fully validated. Because of the lack of validated immunoassays, key to understand risk, epidemiological factors, pathogenesis and mortality, the present inventors developed fusion proteins that comprise nucleocapsid molecular designs aimed at being a reagent in SARS-CoV-2 immunoassays and serological screenings.

The Nucleocapsid (N) protein of SARS-CoV-2 plays a key role in virion assembly through its interaction with the viral genome and membrane protein M. It is an RNA-binding phosphoprotein that can be divided into three parts: an N-terminal RNA-binding domain (NTD), a disordered central Ser/Arg region called aggregation domain (SR), and a C-terminal dimerization domain (CTD) (FIG. 1). The central region takes its name from a Ser and Arg rich sequence that has been suggested to cause nucleocapsid aggregation or self-association.

The inventors of the present invention have developed nucleocapsid fusion proteins lacking the SR aggregation sequence that have surprisingly resulted in reduced ability to self-associate while still being recognized by anti-nucleocapsid antibodies. Said nucleocapsid fusion proteins can be used as key reagents in serological tests for detection of SARS-CoV-2.

The present invention encompass said fusion proteins and the methods for producing thereof, as well as the nucleic acid molecules encoding said fusion proteins, their expression vectors and host cells; it also covers RBD truncations.

### SUMMARY

In a first aspect, the present invention relates to a fusion protein comprising a SARS-CoV-2 nucleocapsid N-terminal domain and/or a SARS-CoV-2 nucleocapsid C-terminal domain, wherein said fusion protein lacks the SARS-CoV-2 nucleocapsid aggregation domain.

In some embodiments, said fusion protein further comprises at least one linker. In some preferred embodiments, said at least one linker is a flexible linker having the amino acid sequence set forth in SEQ ID NO: 5 or SEQ ID NO: 6.

In some embodiments, said fusion protein further comprises a polyhistidine tag. In some preferred embodiments, said polyhistidine tag consists of 6, 8 or 10 histidine residues. In more preferred embodiments, said polyhistidine tag consists of 10 histidine residues having the amino acid sequence set forth in SEQ ID NO: 7.

In some embodiments, said fusion protein further comprises a protease cleavage site. In some preferred embodiments, said protease cleavage site is a tobacco etch virus cleavage site (TEV). In more preferred embodiments, the amino acid sequence of the tobacco etch virus cleavage site (TEV) is set forth in SEQ ID NO: 8.

In some embodiments of the fusion protein of the present invention the aggregation domain is replaced by the flexible linker.

In some embodiments, the nucleocapsid N-terminal domain of the fusion protein has an amino acid sequence of at least 90% sequence identity with SEQ ID NO: 1. In other embodiments, the amino acid sequence of said nucleocapsid N-terminal domain is SEQ ID NO: 1.

In some embodiments, the nucleocapsid C-terminal domain of the fusion protein has an amino acid sequence of at least 90% sequence identity with SEQ ID NO: 2. In other embodiments, the amino acid sequence of the nucleocapsid C-terminal domain is SEQ ID NO: 2.

In some embodiments of the fusion protein of the present invention the nucleocapsid C-terminal domain comprises a Nuclear Localization Signal (NLS). In some preferred embodiments, the amino acid sequence of the Nuclear Localization Signal (NLS) is set forth in SEQ ID NO: 3.

In some embodiments of the present invention, the fusion protein has an amino acid sequence of at least 90% sequence identity with SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17.

In other embodiments of the present invention, the fusion protein comprises the amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

In a further aspect, the present invention relates to a cell comprising the fusion protein described herein.

The present invention also relates to a nucleic acid comprising a nucleotide sequence encoding the fusion protein described herein, a promoter operably linked to the nucleotide sequence and a selectable marker. The present invention also relates to a cell comprising said nucleic acid.

In a further aspect, the present invention relates to a composition comprising the fusion protein described herein, and a solid support, wherein the fusion protein is covalently or non-covalently bound to the solid support.

### BRIEF DESCRIPTION OF THE DRAWINGS

FIG. 1 shows the amino acid sequence and structure of the nucleocapsid (N) protein of SARS-CoV-2.

FIG. 2 shows SDS-PAGE of final purified samples. Samples are reduced (R) or non-reduced (NR) and were run on 4-20% TGX Stain Free gels. M: Protein Ladder (Precision Plus Unstained Protein Standard).

FIG. 3 shows the self-association of nucleocapsid fusion proteins showed by Enzyme-Linked Immuno Sorbent Assay (ELISA).

### DETAILED DESCRIPTION

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Each embodiment in this specification is to be applied mutatis mutandis to every other embodiment unless expressly stated otherwise.

The following terms, unless otherwise indicated, shall be understood to have the following meanings:

As used herein, the term “nucleic acid” refers to any materials comprised of DNA or RNA. Nucleic acids can be made synthetically or by living cells.

As used herein, the term “protein” or refers to large biological molecules, or macromolecules, consisting of one or more chains of amino acid residues. Many proteins are enzymes that catalyze biochemical reactions and are vital to metabolism. Proteins also have structural or mechanical functions, such as actin and myosin in muscle and the proteins in the cytoskeleton, which form a system of scaffolding that maintains cell shape. Other proteins are important in cell signaling, immune responses, cell adhesion, and the cell cycle. However, proteins may be completely artificial or recombinant, i.e., not existing naturally in a biological system.

As used herein, the term “polypeptide” refers to both naturally-occurring and non-naturally-occurring proteins, and fragments, mutants, derivatives and analogs thereof. A polypeptide may be monomeric or polymeric. A polypeptide may comprise a number of different domains (peptides) each of which has one or more distinct activities.

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

As used herein, the term “fusion protein” refers to proteins comprising two or more amino acid sequences that do not co-exist in naturally-occurring proteins. A fusion protein may comprise two or more amino acid sequences from the same or from different organisms. The two or more amino acid sequences of a fusion protein are typically in frame without stop codons between them and are typically translated from mRNA as part of the fusion protein.

The term “fusion protein” and the term “recombinant” can be used interchangeably herein.

As used herein, the term “antigen” refers to a biomolecule that binds specifically to the respective antibody. An antibody from the diverse repertoire binds a specific antigenic structure by means of its variable region interaction.

The terms “antibody” or “immunoglobulin”, as used herein, have the same meaning, and will be used equally in the present invention. The term “antibody” as used herein refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments or derivatives.

The term “binding affinity”, as used herein, refers to the strength of interaction between an antigen's epitope and an antibody's antigen binding site.

As used herein, a “promoter” is a specific nucleic acid sequence that is recognized by a DNA-dependent RNA polymerase (“transcriptase”) as a signal to bind to the nucleic acid and begin the transcription of RNA at a specific site.

The terms “modified sequence” and “modified genes” are used interchangeably herein to refer to a sequence that includes a deletion, insertion or interruption of naturally occurring nucleic acid sequence. In some preferred embodiments, the expression product of the modified sequence is a truncated protein (e.g., if the modification is a deletion or interruption of the sequence). In some particularly preferred embodiments, the truncated protein retains biological activity. In alternative embodiments, the expression product of the modified sequence is an elongated protein (e.g., modifications comprising an insertion into the nucleic acid sequence). In some embodiments, an insertion leads to a truncated protein (e.g., when the insertion results in the formation of a stop codon). Thus, an insertion may result in either a truncated protein or an elongated protein as an expression product.

As used herein, the terms “mutant sequence” and “mutant gene” are used interchangeably and refer to a sequence that has an alteration in at least one codon occurring in a host cell's wild-type sequence. The expression product of the mutant sequence is a protein with an altered amino acid sequence relative to the wild-type. The expression product may have an altered functional capacity (e.g., enhanced binding affinity).

The term “fragment” as used herein, refers to a portion of an amino acid sequence wherein said portion is smaller than the entire amino acid sequence.

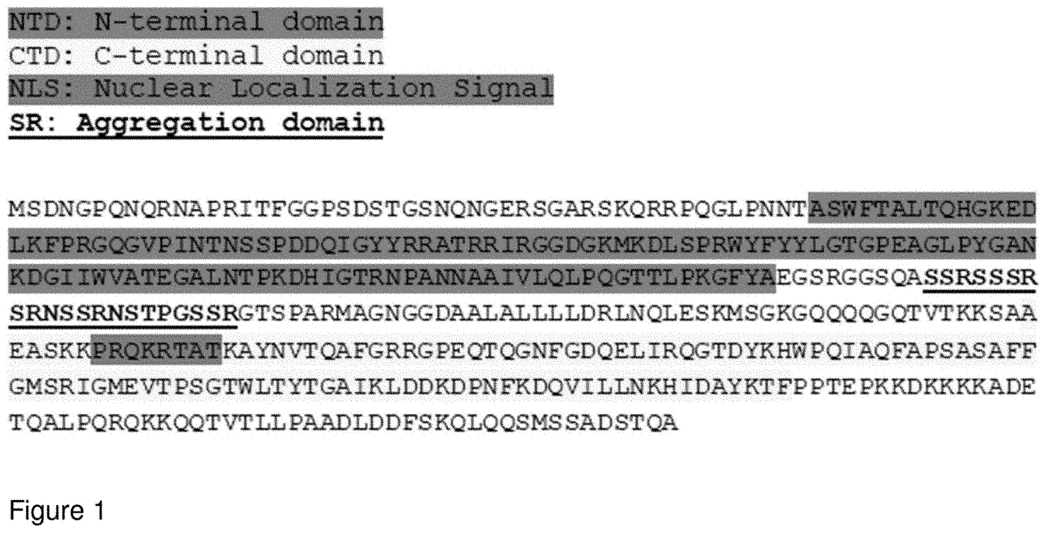

As used herein, the term “nucleocapsid” refers to one of the structural proteins in SARS-CoV-2, which interacts with the viral genome and the membrane protein M. Said nucleocapsid comprises a N-terminal domain (also called NTD) and a C-terminal domain (also called CTD). The structure and amino acid sequence of an exemplary SARS-CoV-2 nucleocapsid protein is shown in FIG. 1.

As used herein, the term “Nuclear Localization Signal” (NLS) refers to a short amino acid sequence that is comprised within the SARS-CoV-2 nucleocapsid protein, more specifically within its C-terminal domain, that acts as a signal for import of the nucleocapsid protein into the cell nucleus.

As used herein, the term “aggregation domain” refers to a disordered central Ser/Arg region of the SARS-CoV-2 nucleocapsid protein.

As used herein, the term “N-terminal signal peptide” is a short peptide (usually 10-30 amino acids long) present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. They are a kind of target peptide.

As used herein, the term “purification tag” or “affinity tag” refer to a polypeptide used to purify proteins that simplifies purification and enables use of standard protocols. In the present invention, the purification tag is a polyhistidine tag of 4, 6, 7, 8, 9, 10, 11 or 12 histidine residues. Preferably, the histidine tag has 6, 8 or 10 histidine residues.

As used herein, the term “linker” refers to a polypeptide comprising of 1-10 amino acids, preferably 3-6 amino acids. The amino acids of the linker may be selected from the group consisting of leucine (Leu, L), isoleucine (Ile, I), alanine (Ala, A), glycine (Gly, G), valine (Val, V), proline (Pro, P), lysine (Lys, K), arginine (Arg, R), Serine (Ser, S), asparagine (Asn, N), and glutamine (Gln, Q), tryptophan (Trp, W), methionine (Met, M), aspartic acid (Asp, D), cysteine (Cys, C), glutamic acid (Glu, E), histidine (His, H), phenylalanine (Phe, F), threonine (The, T), and tyrosine (Tyr, Y). In some preferred embodiments, the linker is a flexible linker that may consist of a sequence of consecutive amino acids that typically include at least one glycine and at least one serine. Exemplary flexible linkers include the amino acid sequences set forth in SEQ ID NO: 5 (GGGS) or SEQ ID NO: 6 (GGSGGGGS), although the precise amino acid sequence of the linker is not particularly limiting.

As used herein, the term “tobacco etch virus cleavage site” or “TEV” refers to a highly site-specific cysteine protease that can be used in fusion proteins as the ones described herein. Its optimal temperature for cleavage is 30° C. but it can also be used at temperature as low as 4° C. Tobacco etch virus cleavage site allows for cleavage of the different domains of a fusion protein of interest. The recognition site for this cysteine protease is the sequence Glu-Asn-Leu-Tyr-Phe-Gln-(Gly/Ser) [ENLYFQ(G/S)] and cleavage occurs between the Gln and Gly/Ser residues. The most commonly used sequence is ENLYFQG. In most cases, the protease is used to cleave affinity tags from fusion proteins.

The term “horseradish peroxidase” or “HRP” is used extensively in biochemistry applications. It is a metalloenzyme with many isoforms, of which the most studied type is C. It catalyzes the oxidation of various organic substrates by hydrogen peroxide.

The term “diagnostic” or “diagnosed”, as used herein, means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

### I. Fusion Proteins

The present invention relates to a fusion protein comprising a SARS-CoV-2 nucleocapsid N-terminal domain and/or a SARS-CoV-2 nucleocapsid C-terminal domain and lacks the SARS-CoV-2 nucleocapsid aggregation domain.

An exemplary amino acid sequence of the CoV-2 nucleocapsid protein is set forth in SEQ ID NO: 9. In some embodiment, the amino acid sequence of the fusion protein of the present invention has between 50% and 90% sequence identity with the sequence set forth in SEQ ID NO: 9. In some embodiments, the fusion protein of the present invention comprises at least a fragment or domain of the nucleocapsid protein set forth in SEQ ID NO: 9. In some preferred embodiments, said fragment or domain of the nucleocapsid protein shares at least 70% of sequence identity with the corresponding fragment in the nucleocapsid protein set forth in SEQ ID NO: 9. More preferably at least 75%, at least 80%, at least 85%, at least 90% or at least 95%.

In some embodiments, the fusion protein of the present invention comprises a SARS-CoV-2 nucleocapsid N-terminal domain. In other embodiments, the fusion protein of the present invention comprises a SARS-CoV-2 nucleocapsid C-terminal domain. In other embodiments, the fusion protein of the present invention comprises a SARS-CoV-2 nucleocapsid N-terminal domain and a SARS-CoV-2 nucleocapsid C-terminal domain.

In some embodiments, the fusion protein of the present invention comprises a SARS-CoV-2 nucleocapsid N-terminal domain having an amino acid sequence of at least 90% sequence identity with SEQ ID NO: 1. In other embodiments, the amino acid sequence of the N-terminal domain has at least 95% identity with SEQ ID NO: 1. In other embodiments, the amino acid sequence of the N-terminal domain has at least 98% identity with SEQ ID NO: 1.

In some embodiments, the fusion protein of the present invention comprises a SARS-CoV-2 nucleocapsid C-terminal domain having an amino acid sequence of at least 90% sequence identity with SEQ ID NO: 2. In other embodiments, the amino acid sequence of the C-terminal domain has at least 95% identity with SEQ ID NO: 2. In other embodiments, the amino acid sequence of the C-terminal domain has at least 98% identity with SEQ ID NO: 2.

In other embodiments, the fusion protein of the present invention comprises a SARS-CoV-2 nucleocapsid N-terminal domain having an amino acid sequence of at least 90% sequence identity with SEQ ID NO: 1 and a SARS-CoV-2 nucleocapsid C-terminal domain having an amino acid sequence of at least 90% sequence identity with SEQ ID NO: 2. In more preferred embodiments, the fusion protein of the present invention comprises a SARS-CoV-2 nucleocapsid N-terminal domain having an amino acid sequence of at least 95% sequence identity with SEQ ID NO: 1 and a SARS-CoV-2 nucleocapsid C-terminal domain having an amino acid sequence of at least 95% sequence identity with SEQ ID NO: 2.

In some embodiments, the nucleocapsid C-terminal domain of the fusion protein of the present invention comprises a Nuclear Localization Signal (NLS). In more preferred embodiments, the amino acid sequence of Nuclear Localization Signal (NLS) is set forth in SEQ ID NO: 3.

The fusion proteins of the present invention can be obtained by methods well-known to the skilled person. For example, said fusion proteins can be obtained recombinantly in bacteria, yeasts, fungi, or mammalian cells. In one embodiment, the fusion proteins of the present invention are produced in prokaryotic cells, such as Escherichia coli, but other prokaryotic cells can be used. In another embodiment, the fusion proteins of the present invention are produced in eukaryotic cells, such as human embryotic kidney (HEK) or Chinese hamster ovary (CHO) cells, but other eukaryotic cells can be used.

The fusion proteins of the present invention can be purified from the cells by methods well known to the skilled person. Said methods include, without limitation, filtration, conjugation, affinity chromatography, ion exchange chromatography, hydrophobic interaction chromatography, and size exclusion chromatography.

The fusion proteins of the present invention may further comprise at least one linker. As previously described, linkers are polypeptides comprising of 1-10 amino acids, preferably 3-6 amino acids. In some preferred embodiment, the linkers of the fusion proteins of the present invention are flexible linkers that may improve the tolerance for assembly of the different domains of said fusion proteins, and are often a combination of glycine and serine residues. However, it is not obvious to the skilled person if the inclusion of the selected linkers would result in functional fusion proteins. In one embodiment, said linker is a flexible linker. In more preferred embodiments, the flexible linker has the amino acid sequence set forth in SEQ ID NO: 5 or SEQ ID NO: 6.

In some embodiments, the fusion proteins of the present invention comprise at least one flexible linker. In other embodiments, said fusion proteins comprise at least two flexible linkers. In some embodiments, the flexible linker is placed in the position of the aggregation domain. In other embodiments, the aggregation domain is replaced by the flexible linker. In other embodiments, the aggregation domain is replaced by at least a flexible linker.

The fusion proteins of the present invention may further comprise a polyhistidine tag. As previously described, the use of purification or affinity tags simplifies purification and enables use of standard protocols in the production of fusion proteins. For example, the histidine (His) tag (also known as polyhistidine or polyHis) is known to be useful, for example, in the purification by Immobilized Metal Affinity Chromatography (IMAC). Other uses of the polyhistidine tag are also well-known by the skilled person and therefore the polyhistidine tag of the present invention is not limited to the purification functionality. In the present invention, said polyhistidine tag consists of 6, 8 or 10 histidine residues, although other histidine (his) tags comprising 7, 9, 11 or 12 histidine residues are also possible. In some preferred embodiments, the polyhistidine tag of the fusion proteins of the present invention has the amino acid sequence set forth in SEQ ID NO: 7.

The fusion proteins of the present invention may further comprise a protease cleavage site. In preferred embodiments, said protease cleavage site is a tobacco etch virus cleavage site (TEV). As previously described, the use of a tobacco etch virus cleavage site allows for cleavage of the different domains of a fusion protein of interest. In some preferred embodiments, the amino acid sequence of the tobacco etch virus cleavage site (TEV) is set forth in SEQ ID NO: 8.

### II. Exemplary Fusion Proteins

In some embodiments, the fusion protein of the present invention has an amino acid sequence of at least 90% sequence identity with SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17. In other embodiments, the fusion protein of the present invention has an amino acid sequence of at least 95% sequence identity with SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17. In other embodiments, the fusion protein of the present invention has an amino acid sequence of at least 98% sequence identity with SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17.

In more preferred embodiments, the fusion protein of the present invention comprises the amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

### III. Nucleic Acids, Cloning Cells, and Expression Cells

The present invention also relates to nucleic acids comprising a nucleotide sequence encoding the fusion proteins described herein. The nucleic acid may be DNA or RNA. DNA comprising a nucleotide sequence encoding a fusion protein described herein typically comprises a promoter that is operably-linked to the nucleotide sequence. The promoter is preferably capable of driving constitutive or inducible expression of the nucleotide sequence in an expression cell of interest. Said nucleic acid may also comprise a selectable marker useful to select the cell containing said nucleic acid of interest. Useful selectable markers are well known by the skilled person. The precise nucleotide sequence of the nucleic acid is not particularly limiting so long as the nucleotide sequence encodes a fusion protein described herein. Codons may be selected, for example, to match the codon bias of an expression cell of interest (e.g., a mammalian cell such as a human cell) and/or for convenience during cloning. DNA may be a plasmid, for example, which may comprise an origin of replication (e.g., for replication of the plasmid in a prokaryotic cell).

In one embodiment described herein, the present invention refers to a nucleic acid comprising a nucleotide sequence encoding the fusion protein, a promoter operably linked to the nucleotide sequence and a selectable marker.

Various aspects of the present invention also relate to a cell comprising a nucleic acid comprising a nucleotide sequence that encodes a fusion protein as described herein. The cell may be an expression cell or a cloning cell. Nucleic acids are typically cloned in E. coli, although other cloning cells may be used.

If the cell is an expression cell, the nucleic acid is optionally a nucleic acid of a chromosome, i.e., wherein the nucleotide sequence is integrated into the chromosome, although then nucleic acid may be present in an expression cell, for example, as extrachromosomal DNA or vectors, such as plasmids, cosmids, phages, etc. The format of the vector should not be considered limiting.

In one embodiment described herein, the cell is typically an expression cell. The nature of the expression cell is not particularly limiting. Expression cells which may be used are prokaryotic cells such as E. coli and Bacillus spp. and eukaryotic cells such as yeast cells (e.g. S. cerevisiae, S. pombe, P. pastoris, K lactis, H polymorpha), insect cells (e.g. Sf9), fungal, plant cells or mammalian cells. Mammalian expression cells may allow for favorable folding, post-translational modifications, and/or secretion of a fusion protein, although other eukaryotic cells or prokaryotic cells may be used as expression cells. Exemplary expression cells include TunaCHO, ExpiCHO, Expi293, BHK, NS0, Sp2/0, COS, C127, HEK, HT-1080, PER.C6, HeLa, and Jurkat cells. The cell may also be selected for integration of a vector, more preferably for integration of a plasmid DNA.

In some preferred embodiments described herein, the cell is typically an expression cell. In more preferred embodiments, the expression cell is Escherichia coli, but other expression cells can also be used.

The fusion proteins of the present invention can be produced by appropriate transfection strategy of the nucleic acids comprising a nucleotide sequence that encodes the fusion proteins into prokaryotic or eukaryotic cells. The skilled person is aware of the different techniques available for transfection of nucleic acids into the cell line of choice (lipofection, electroporation, etc). Thus, the choice of the prokaryotic or eukaryotic cell line or species and transfection strategy should not be considered limiting. The cell line could be further selected for integration of the plasmid DNA.

Various aspects of the present invention also relate to a cell comprising the fusion proteins described herein.

### IV. Compositions and Methods Related to Assays

Various aspects of the present invention relate to compositions comprising a fusion protein as described herein. In some embodiments, the composition may comprise a pharmaceutically-acceptable carrier and/or a pharmaceutically-acceptable excipient. The composition may be, for example, a vaccine.

Various embodiments of the present invention relate to a method of treating or preventing a SARS-CoV-2 infection in a human patient comprising administering to the patient a composition comprising a fusion protein as described herein. The term “preventing” as used herein refers to prophylaxis, which includes the administration of a composition to a patient to reduce the likelihood that the patient will become infected with SARS-CoV-2 relative to an otherwise similar patient who does not receive the composition. The term preventing also includes the administration of a composition to a group of patients to reduce the number of patients in the group who become infected with SARS-CoV-2 relative to an otherwise similar group of patients who do not receive the composition.

Various embodiments of the invention relate to a method of treating or preventing a SARS-CoV-2 infection in a human patient comprising administering to the patient a vaccine according to the embodiments described herein.

A patient may be infected with SARS-CoV-2, a patient may have been exposed to SARS-CoV-2, or a patient may present with an elevated risk for exposure to and/or infection with SARS-CoV-2.

In some embodiments described herein, the composition comprises the fusion protein of the present invention and a solid support.

In other embodiments, the composition comprises the fusion protein of the present invention and a solid support, wherein the fusion protein is covalently or non-covalently bound to the solid support. The term “non-covalently bound,” as used herein, refers to specific binding such as between an antibody and its antigen, a ligand and its receptor, or an enzyme and its substrate, exemplified, for example, by the interaction between streptavidin binding protein and streptavidin or an antibody and its antigen.

In other embodiments, the composition comprises the fusion protein of the present invention and a solid support, wherein the fusion protein is directly or indirectly bound to a solid support. The term “direct” binding, as used herein, refers to the direct conjugation of a molecule to a solid support, e.g., a gold-thiol interaction that binds a cysteine thiol of a fusion protein to a gold surface. The term “indirect” binding, as used herein, includes the specific binding of a fusion protein to another molecule that is directly bound to a solid support, e.g., a fusion protein may bind an antibody that is directly bound to a solid support thereby indirectly binding the fusion protein to the solid support. The term “indirect” binding is independent of the number of molecules between the fusion protein and the solid support so long as (a) each interaction between the daisy chain of molecules is a specific or covalent interaction and (b) a terminal molecule of the daisy chain is directly bound to the solid support.

A solid support may comprise a particle, a bead, a membrane, a surface, a polypeptide chip, a microtiter plate, or the solid-phase of a chromatography column.

A composition may comprise a plurality of beads or particles, wherein each bead or particle of the plurality of beads or particles are directly or indirectly bound to at least one fusion protein as described herein. A composition may comprise a plurality of beads or particles, wherein each bead or particle of the plurality of beads or particles are covalently or non-covalently bound to at least one fusion protein as described herein.

Various aspects of the present invention relate to a kit for detecting the presence of antibodies against the fusion proteins of the present invention, and/or fragment thereof in a sample, said kit comprising a fusion protein and a solid support or composition as described herein.

The compositions and kits described herewith can be either for use in an assay or in compositions that are generated during the performance of an assay. Various aspects of the invention relate to a diagnostic medical device comprising a composition as described herein.

Various aspects of the invention relate to assays for detection of anti-SARS-CoV-2 antibodies.

Assays typically feature a solid support that either allows for measurement, such as by turbidimetry, nephelometry, UV/Vis/IR spectroscopy (e.g., absorption, transmission), fluorescence or phosphorescence spectroscopy, or surface plasmon resonance, or aids in the separation of components that directly or indirectly bind the solid support from components that do not directly or indirectly bind the solid support, or both. For example, an assay may include a composition comprising particles or beads and/or that aid in the mechanical separation of components that directly or indirectly bind the particles or beads.

Other exemplary assays that may include the fusion protein or the composition of the present invention includes but it is not limited to ELISA, lateral flow, single molecule counting (SMC), viscoelastic tests such as Sonoclot, gel technologies, fluorescence assay and other point-of-care testing using any of these techniques.

The fusion proteins, compositions, kits and the like for detection of SARS-CoV-2 as described herein are further illustrated by the following non-limiting examples.

### EXAMPLES

### Example 1: Expression and Purification of the Fusion Proteins of the Present Invention

The nucleocapsid fusion proteins of the present invention were produced in Escherichia coli BL21 (DE3) cells and affinity purified from the supernatant of the lysed cells. The affinity purification was carried out according to IMAC standard protocols that include imidazole washes and elution. After spin concentration, the proteins were subjected to a size exclusion polishing step and purity evaluation by SDS-PAGE.

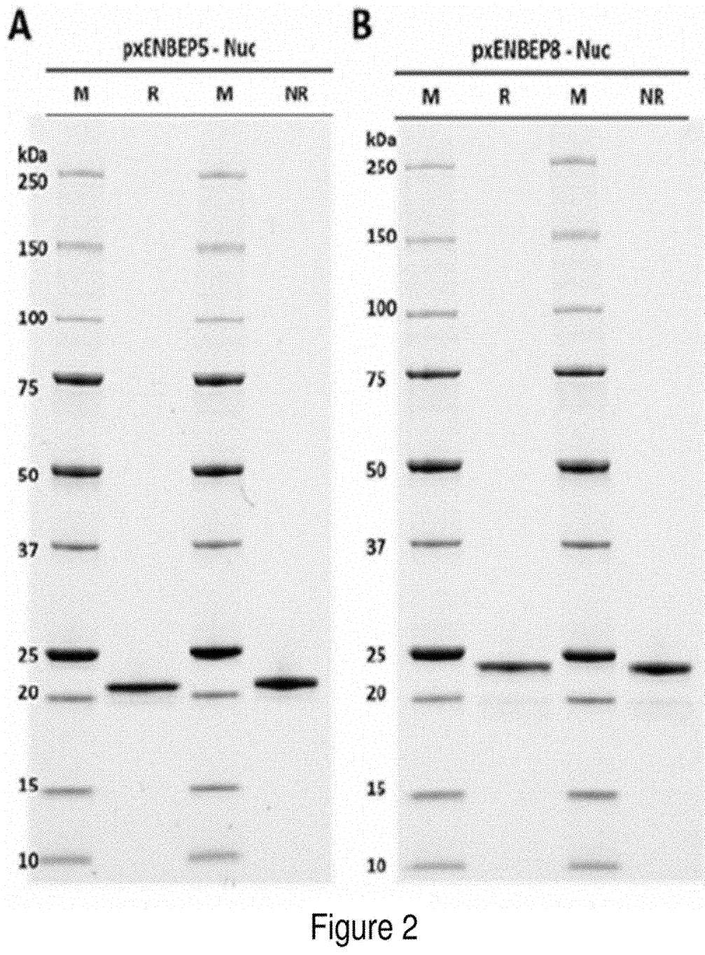

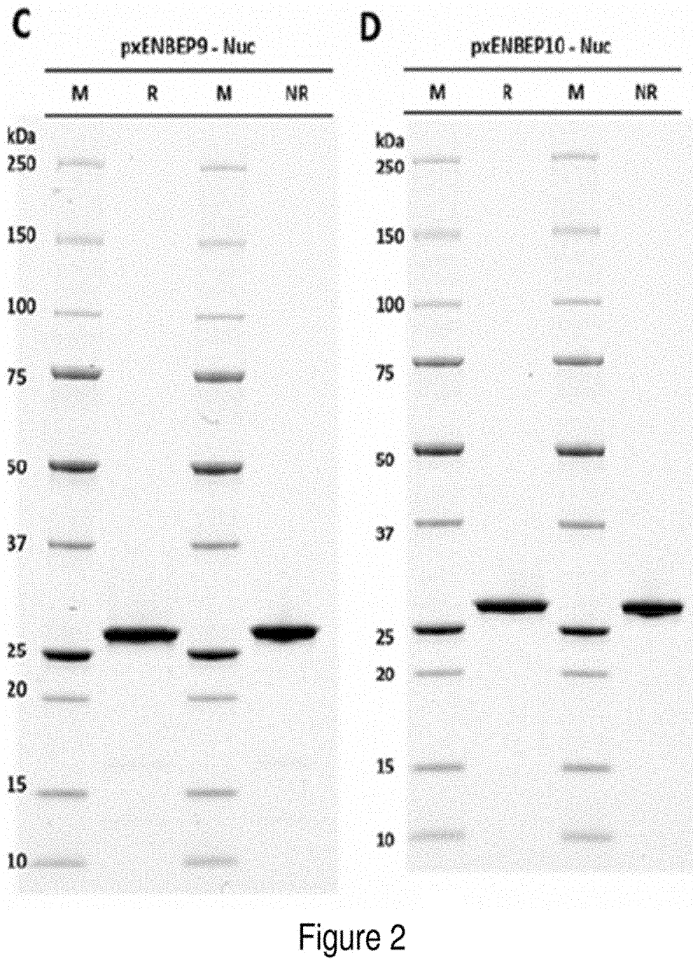

FIG. 2 shows final purified samples characterized by SDS-PAGE for some of the fusion proteins. Table 1 includes the molecular weight of the final products measured by intact mass spectrometry.

Thus, the nucleocapsid fusion proteins of the present invention have been expressed, purified and characterized.

TABLE 1
Final Molecular Weight measured by Intact Mass Spectrometry

| | Theoretical | Measured | |
|---|---|---|---|
| Construct | MW (Da) | MW (Da) | Comments |
| pxENBEP3 - Nuc | 45549 | N/A | N/A |
| pxENBEP5 - Nuc | 21306 | 21175.9 | Loss of initial Met |
| pxENBEP8 - Nuc | 21477 | 21347.2 | Loss of initial Met |
| pxENBEP9 - Nuc | 27297 | 27167.1 | Loss of initial Met |
| pxENBEP10 - Nuc | 28092 | 27961.8 | Loss of initial Met |

### Example 2: Antibody Recognition and Self-Association of the Nucleocapsid Fusion Proteins of the Present Invention

All of the nucleocapsid fusion proteins of the present invention were recognized by anti-nucleocapsid polyclonal antibodies (not-shown). Constructs that included both the NTD (N-terminal domain) and CTD (C-terminal domain) showed slightly stronger signals than constructs that only included either the NTD or the CTD.

| Assay | | | Detection |
|---|---|---|---|
| format | Coating | Primary | antibody |
| 1 | Biotinylated - | 1% BSA, PBS-T | Strep-Tag HRP |
| | nucleocapsid | | |
| 2 | Nucleocapsid | Biotinylated - | Strep-Tag HRP |
| | | nucleocapsid | |

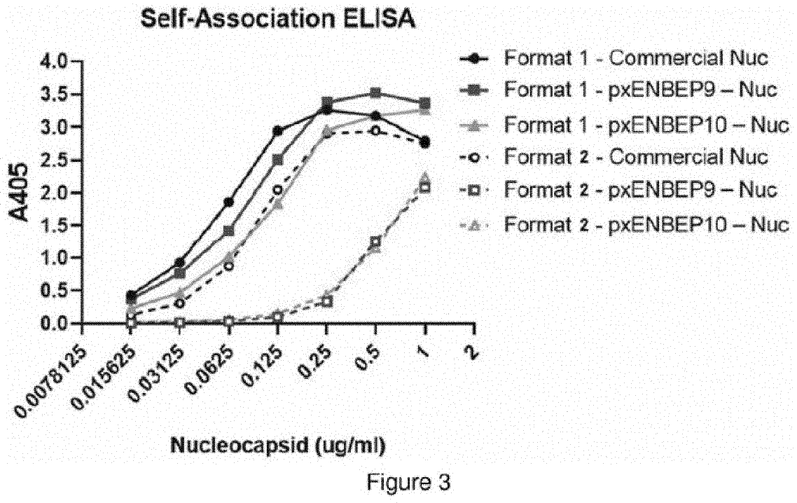

The ability of the nucleocapsid fusion proteins to self-associate was evaluated by ELISA. Briefly, the different nucleocapsid fusion proteins were coated on the plate overnight at 4° C. After wash and BSA blocking, biotinylated-nucleocapsid was added and incubated for 1 h while shaking. The level of self-association was visualized by adding anti Strep-Tag HRP, which recognizes biotinylated proteins. Coating with biotinylated proteins (Assay Format 1) was used as a control to show that all proteins are equally recognized by Anti-Strep HRP. As shown in FIG. 3, both pxENBEP9-Nuc (SEQ ID NO: 16) and pxENBEP10-Nuc (SEQ ID NO: 17) exhibited a lower level of self-association, with the signal dropping off considerably faster than with commercially available full length nucleocapsid.

SEQUENCES

| SEQ ID | | |
|---|---|---|
| NOS | Sequence (5′ to 3′) | Comments |
| SEQ ID NO: 1 | ASWFTALTQHGKEDLKFPRGQGVPINTNSSPDD | Nucleocapsid |
| | QIGYYRRATRRIRGGDGKMKDLSPRWYFYYLGT | N-terminal |
| | GPEAGLPYGANKDGIIWATEGALNTPKDHIGTR | domain |
| | NPANNAAIVLQLPQGTTLPKGFYA | |
| SEQ ID NO: 2 | AEASKKPRQKRTATKAYNVTQAFGRRGPEQTQG | Nucleocapsid |
| | NFGDQELIRQGTDYKHWPQIAQFAPSASAFFGMS | C-terminal |
| | RIGMEVTPSGTWLTYTGAIKLDDKDPNFKDQVILL | domain |
| | NKHIDAYKTF | |
| SEQ ID NO: 3 | PRQKRTAT | Nuclear |
| | | Localization |
| | | Signal |
| SEQ ID NO: 4 | SSRSSSRSRNSSRNSTPGSSR | Aggregation |
| | | domain |
| SEQ ID NO: 5 | GGGS | Flexible linker |
| SEQ ID NO: 6 | GGSGGGGS | Flexible linker |
| SEQ ID NO: 7 | HHHHHHHHHH | His tag (10x) |
| SEQ ID NO: 8 | ENLYFQ | TEV Cleavage |
| | | site |
| SEQ ID NO: 9 | MSDNGPQNQRNAPRITFGGPSDSTGSNQNGERS | Nucleocapsid |
| | GARSKQRRPQGLPNNTASWFTALTQHGKEDLKF | protein |
| | PRGQGVPINTNSSPDDQIGYYRRATRRIRGGDGK | |
| | MKDLSPRWYFYYLGTGPEAGLPYGANKDGIIWVA | |
| | TEGALNTPKDHIGTRNPANNAAIVLQLPQGTTLPK | |
| | GFYAEGSRGGSQASSRSSSRSRNSSRNSTPGSS | |
| | RGTSPARMAGNGGDAALALLLLDRLNQLESKMS | |
| | GKGQQQQGQTVTKKSAAEASKKPRQKRTATKAY | |
| | NVTQAFGRRGPEQTQGNFGDQELIRQGTDYKHW | |
| | PQIAQFAPSASAFFGMSRIGMEVTPSGTWLTYTG | |
| | AIKLDDKDPNFKDQVILLNKHIDAYKTFPPTEPKKD | |
| | KKKKADETQALPQRQKKQQTVTLLPAADLDDFSK | |
| | QLQQSMSSADSTQA | |
| SEQ ID | MSDNGPQNQRNAPRITFGGPSDSTGSNQNGERS | pxENBEP3 - Nuc |
| NO: 10 | GARSKQRRPQGLPNNTASWFTALTQHGKEDLKF | |
| | PRGQGVPINTNSSPDDQIGYYRRATRRIRGGDGK | |
| | MKDLSPRWYFYYLGTGPEAGLPYGANKDGIIWVA | |
| | TEGALNTPKDHIGTRNPANNAAIVLQLPQGTTLPK | |
| | GFYAEGSRGGSQAGGSGGGGSGTSPARMAGN | |
| | GGDAALALLLLDRLNQLESKMSGKGQQQQGQTV | |
| | TKKSAAEASKKPRQKRTATKAYNVTQAFGRRGP | |
| | EQTQGNFGDQELIRQGTDYKHWPQIAQFAPSAS | |
| | AFFGMSRIGMEVTPSGTWLTYTGAIKLDDKDPNF | |
| | KDQVILLNKHIDAYKTFPPTEPKKDKKKKADETQA | |
| | LPQRQKKQQTVTLLPAADLDDFSKQLQQSMSSA | |
| | DSTQAGGGSHHHHHHHHHH | |
| SEQ ID | MSHHHHHHHHHHGGGSENLYFQMSDNGPQNQ | pxENBEP4 - Nuc |
| NO: 11 | RNAPRITFGGPSDSTGSNQNGERSGARSKQRRP | |
| | QGLPNNTASWFTALTQHGKEDLKFPRGQGVPINT | |
| | NSSPDDQIGYYRRATRRIRGGDGKMKDLSPRWY | |
| | FYYLGTGPEAGLPYGANKDGIIWATEGALNTPK | |
| | DHIGTRNPANNAAIVLQLPQGTTLPKGFYAEGSR | |
| | GGSQAGGSGGGGSGTSPARMAGNGGDAALALL | |
| | LLDRLNQLESKMSGKGQQQQGQTVTKKSAAEAS | |
| | KKPRQKRTATKAYNVTQAFGRRGPEQTQGNFGD | |
| | QELIRQGTDYKHWPQIAQFAPSASAFFGMSRIGM | |
| | EVTPSGTWLTYTGAIKLDDKDPNFKDQVILLNKHI | |
| | DAYKTFPPTEPKKDKKKKADETQALPQRQKKQQT | |
| | VTLLPAADLDDFSKQLQQSMSSADSTQA | |
| SEQ ID | MSDNGPQNQRNAPRITFGGPSDSTGSNQNGERS | pxENBEP5 - Nuc |
| NO: 12 | GARSKQRRPQGLPNNTASWFTALTQHGKEDLKF | |
| | PRGQGVPINTNSSPDDQIGYYRRATRRIRGGDGK | |
| | MKDLSPRWYFYYLGTGPEAGLPYGANKDGIIWVA | |
| | TEGALNTPKDHIGTRNPANNAAIVLQLPQGTTLPK | |
| | GFYAEGSRGGSQAGGGSHHHHHHHHHH | |
| SEQ ID | MSHHHHHHHHHHGGGSENLYFQMSDNGPQNQ | pxENBEP6 - Nuc |
| NO: 13 | RNAPRITFGGPSDSTGSNQNGERSGARSKQRRP | |
| | QGLPNNTASWFTALTQHGKEDLKFPRGQGVPINT | |
| | NSSPDDQIGYYRRATRRIRGGDGKMKDLSPRWY | |
| | FYYLGTGPEAGLPYGANKDGIIWATEGALNTPK | |
| | DHIGTRNPANNAAIVLQLPQGTTLPKGFYAEGSR | |
| | GGSQA | |
| SEQ ID | MSAEASKKPRQKRTATKAYNVTQAFGRRGPEQT | pxENBEP7 - Nuc |
| NO: 14 | QGNFGDQELIRQGTDYKHWPQIAQFAPSASAFFG | |
| | MSRIGMEVTPSGTWLTYTGAIKLDDKDPNFKDQVI | |
| | LLNKHIDAYKTFPPTEPKKDKKKKADETQALPQRQ | |
| | KKQQTVTLLPAADLDDFSKQLQQSMSSADSTQA | |
| | GGGSHHHHHHHHHH | |
| SEQ ID | MSHHHHHHHHHHGGGSENLYFQAEASKKPRQK | pxENBEP8 - Nuc |
| NO: 15 | RTATKAYNVTQAFGRRGPEQTQGNFGDQELIRQ | |
| | GTDYKHWPQIAQFAPSASAFFGMSRIGMEVTPSG | |
| | TWLTYTGAIKLDDKDPNFKDQVILLNKHIDAYKTFP | |
| | PTEPKKDKKKKADETQALPQRQKKQQTVTLLPAA | |
| | DLDDFSKQLQQSMSSADSTQA | |
| SEQ ID | MSASWFTALTQHGKEDLKFPRGQGVPINTNSSP | pxENBEP9 - Nuc |
| NO: 16 | DDQIGYYRRATRRIRGGDGKMKDLSPRWYFYYL | |
| | GTGPEAGLPYGANKDGIIWATEGALNTPKDHIGT | |
| | RNPANNAAIVLQLPQGTTLPKGFYAGGSGGGGS | |
| | AEASKKNVTQAFGRRGPEQTQGNFGDQELIRQG | |
| | TDYKHWPQIAQFAPSASAFFGMSRIGMEVTPSGT | |
| | WLTYTGAIKLDDKDPNFKDQVILLNKHIDAYKTFG | |
| | GGSHHHHHHHHHH | |
| SEQ ID | MSHHHHHHHHHHGGGSENLYFQASWFTALTQH | pxENBEP10 - Nud |
| NO: 17 | GKEDLKFPRGQGVPINTNSSPDDQIGYYRRATRR | |
| | IRGGDGKMKDLSPRWYFYYLGTGPEAGLPYGAN | |
| | KDGIIWATEGALNTPKDHIGTRNPANNAAIVLQL | |
| | PQGTTLPKGFYAGGSGGGGSAEASKKNVTQAFG | |
| | RRGPEQTQGNFGDQELIRQGTDYKHWPQIAQFA | |
| | PSASAFFGMSRIGMEVTPSGTWLTYTGAIKLDDK | |
| | DPNFKDQVILLNKHIDAYKTF | |

1. A fusion protein comprising a SARS-CoV-2 nucleocapsid N-terminal domain and/or a SARS-CoV-2 nucleocapsid C-terminal domain, wherein said fusion protein lacks the SARS-CoV-2 nucleocapsid aggregation domain.

2. The fusion protein, according to claim 1, further comprising at least one linker.

3. The fusion protein, according to claim 2, wherein the at least one linker is a flexible linker having the amino acid sequence set forth in SEQ ID NO: 5 or SEQ ID NO: 6.

4. The fusion protein, according to any of the preceding claims, further comprising a polyhistidine purification tag.

5. The fusion protein, according to claim 4, wherein the polyhistidine tag consists of 6, 8 or 10 histidine residues.

6. The fusion protein, according to claim 5, wherein the polyhistidine tag consists of 10 histidine residues having the amino acid sequence set forth in SEQ ID NO: 7.

7. The fusion protein, according to any of the preceding claims, further comprising a protease cleavage site.

8. The fusion protein, according to claim 7, wherein the protease cleavage site is a tobacco etch virus cleavage site (TEV).

9. The fusion protein, according to claim 8, wherein the amino acid sequence of the tobacco etch virus cleavage site (TEV) is set forth in SEQ ID NO: 8.

10. The fusion protein, according to any of the preceding claims, wherein the aggregation domain is replaced by the flexible linker.

11. The fusion protein, according to any of the preceding claims, wherein the nucleocapsid N-terminal domain has an amino acid sequence of at least 90% sequence identity with SEQ ID NO: 1.

12. The fusion protein, according to claim 11, wherein the amino acid sequence of the nucleocapsid N-terminal domain is SEQ ID NO: 1.

13. The fusion protein, according to any of the preceding claims, wherein the nucleocapsid C-terminal domain has an amino acid sequence of at least 90% sequence identity with SEQ ID NO: 2.

14. The fusion protein, according to claim 13, wherein the amino acid sequence of the nucleocapsid C-terminal domain is SEQ ID NO: 2.

15. The fusion protein, according to any of the preceding claims, wherein the nucleocapsid C-terminal domain comprises a Nuclear Localization Signal (NLS).

16. The fusion protein, according to claim 15, wherein the amino acid sequence of the Nuclear Localization Signal (NLS) is set forth in SEQ ID NO: 3.

17. The fusion protein, according to any of the preceding claims, wherein said fusion protein has an amino acid sequence of at least 90% sequence identity with SEQ ID NO: 10, or SEQ ID NO: 11, or SEQ ID NO: 12, or SEQ ID NO: 13, or SEQ ID NO: 14, or SEQ ID NO: 15, or SEQ ID NO: 16, or SEQ ID NO: 17.

18. The fusion protein, according to any of the preceding claims, wherein said fusion protein comprises the amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, and SEQ ID NO: 17.

19. A cell, comprising the fusion protein according to any one of the preceding claims.

20. A nucleic acid comprising a nucleotide sequence encoding the fusion protein according to any one of claims 1 to 18, a promoter operably linked to the nucleotide sequence and a selectable marker.

21. A cell comprising the nucleic acid of claim 20.

22. A composition comprising the fusion protein of any one of claims 1 to 18, and a solid support, wherein the fusion protein is covalently or non-covalently bound to the solid support.
